(19) United States
(12) Patent Application Publication
(10) Pub. No.: US 2023/0066204 A1
(43) Pub. Date: Mar. 2, 2023

(54) Caffeine Citrate Formulations

(71) Applicant: OYE Therapeutics, Inc, Crown Point, IN (US)

(72) Inventors: Brett Dines, Schererville, IN (US); Pamela A. Smith, West Lafayette, IN (US); Stephen R. Byrn, West Lafayette, IN (US)

(21) Appl. No.: 17/784,375

(22) PCT Filed: Dec. 18, 2020
(86) PCT No.: PCT/US2020/065957

§ 371 Date: Jun. 10, 2022

Related U.S. Application Data
(60) Provisional application No. 62/951,807, filed on Dec. 20, 2019.

(51) Int. Cl. A61K 31/522; A61K 47/12

(52) U.S. Cl. CPC A61K 31/522; A61K 47/12

(57) ABSTRACT

The present disclosure is directed to compositions of caffeine citrate. In addition, the present disclosure includes processes of making compositions of caffeine citrate and methods of treatment using caffeine citrate. Such methods include methods of accelerating emergence from anesthesia by delivering compositions of caffeine citrate of the disclosure.

### CROSS-REFERENCE TO RELATED APPLICATIONS

This application claims benefit of, and priority to, U.S. Provisional Application No. 62/951,807, filed on Dec. 20, 2019, the entire contents of which is specifically incorporated by reference herein.

### BACKGROUND

Anesthetic agents are known to inhibit neurotransmitter release from both neurons and secretory cells. Caffeine has been shown to elevate intracellular cAMP levels which are in turn known to facilitate neurotransmitter release. In rat studies, caffeine was effective in accelerating emergence from anesthesia when administered intravenously. The same has also been shown in humans. In a single-center double-blind two-way crossover trial design participants underwent two sessions of general anesthesia administered a minimum of 2 weeks apart. A 15 mg/kg caffeine citrate infusion (corresponding to 7.5 mg/kg of caffeine base) was given and the study results showed a substantial reduction in mean time to emerge from anesthesia (16.5 ± 3.9 (SD) min when receiving saline and 9.6 ± 5.1 (SD) min when receiving caffeine (P = 0.002)). The following references which relate to caffeine citrate clinical trials are hereby incorporated by reference: U.S National Library of Medicine (2018) (ClinicalTrials.gov Identifier NCT02567968), A Study to Determine if Caffeine Accelerates Emergence from Anesthesia; Wang, Q., Fong, R., Mason, P., Fox, A. and Xie, Z. (2013), Caffeine accelerates recovery from general anesthesia. Journal of Neurophysiology, 111(6), pp.1331-1340; Fong, R., Khokhar, S., Chowdhury, A., Xie, K., Wong, J., Fox, A. and Xie, Z. (2017), Caffeine accelerates recovery from general anesthesia via multiple pathways. Journal of Neurophysiology, 118(3), pp.1591-1597; Fong, R., Wang, L., Zacny, J., Khokhar, S., Apfelbaum, J., Fox, A. and Xie, Z. (2018). Caffeine Accelerates Emergence from Isoflurane Anesthesia in Humans. Anesthesiology, 129(5), pp.912-920; and U.S National Library of Medicine (2017) (ClinicalTrials.gov Identifier NCT03360903), A Study to Determine if Caffeine Accelerates Emergence from Propofol Anesthesia.

At 15 mg/kg, however, a 100 kg patient would require 1500 mg of caffeine citrate. Given the limited solubility of caffeine citrate in water (measured herein) at about 56 mg/ml at room temperature, this would require about 27 ml. It would be preferable to have a formulation of caffeine citrate with a higher solubility so as to reduce the volume of solution required to deliver a pharmaceutically effective dose of caffeine citrate.

### SUMMARY

In one aspect of the disclosure, compositions comprising caffeine citrate and one or more co-solvents are provided.

In a further aspect of the disclosure, methods of making compositions comprising caffeine citrate, water and a co-solvent comprising the steps of combining one or more co-solvents with caffeine citrate are provided, as well as compositions made by those methods.

In an additional aspect of the disclosure, compositions comprising caffeine citrate and one or more of sorbitol, propylene glycol, polyethylene glycol, glycerin, lactic acid, and orthophosphoric acid are provided.

In a still further aspect of the disclosure, solution compositions comprising caffeine citrate, water, and one or more co-solvents wherein the solubility of caffeine citrate in the solution composition is greater than or equal to 100 mg/ml solution are provided.

In a further aspect of the disclosure, methods of delivering a composition of the disclosure to a human patient comprising the steps of preparing a solution of the disclosure and administering said solution to said human are provided.

In yet an additional aspect of the disclosure, methods of accelerating emergence from anesthesia comprising administering to a patient undergoing a medical procedure an anesthesia agent and a pharmaceutically effective amount of a composition of the disclosure are provided.

In a still further aspect of the disclosure, processes for preparing compositions of the disclosure comprising the steps of combining one or more co-solvent solutions with a solution of caffeine citrate are provided.

In additional aspects of the disclosure, compositions comprising lactic acid, water, and caffeine citrate are provided.

In still further aspects of the disclosure, methods for treating apnea, asthma, and/or headaches with compositions comprising caffeine citrate are provided.

In additional aspects of the disclosure, compositions comprising caffeine citrate of the disclosure and one or more pharmaceutically acceptable excipients are provided.

In still further aspects of the disclosure, uses of a caffeine citrate composition of the disclosure for accelerating the emergence from anesthesia are provided.

In additional aspects of the disclosure, uses of a caffeine citrate composition of the disclosure for the preparation of a medicament comprising a therapeutically effective amount of caffeine citrate is provided.

In yet additional aspects of the disclosure, uses of a caffeine citrate composition of the disclosure for the treatment of headaches, asthma, or apnea are provided.

In further aspects of the disclosure, foodstuffs comprising caffeine citrate compositions of the disclosure are provided.

### DETAILED DESCRIPTION

The disclosure is directed to compositions comprising caffeine citrate and one or more co-solvents and the uses of such compositions, for example, in accelerating recovery in patients who are under general anesthesia during medical procedures. The term “co-solvent” refers to a non-water based solvent for which caffeine citrate has some solubility when the co-solvent is a liquid. Thus, one can have a solution with caffeine citrate and a single co-solvent and such compositions are within the scope of the disclosure. The compositions of the disclosure may further, for example, contain water. The compositions of the disclosure are typically solutions.

Examples of co-solvents of the disclosure include alcohols, organic acids, and inorganic acids and in many embodiments, compositions of the disclosure comprise one or more of an organic acid, an inorganic acid, and an alcohol. In these and other embodiments, water may be further added.

Alcohols of the disclosure include primary alcohols such as polyethylene glycol, diols such as propylene glycol, and triols such as glycerin. Examples of alcohols further include sugar alcohols such as sorbitol and more particularly D-(-)-Sorbitol. Examples of organic acids include amino acids and C₁-C₅ acids such as lactic acid. An example of an inorganic acid is phosphoric acid including ortho-phosphoric acid.

The compositions of the disclosure may further be buffered such as with citric acid and sodium citrate. The compositions may further include one or more pharmaceutically acceptable excipients. In some embodiments, the composition of the invention, does not contain citric acid or sodium citrate.

In some embodiments of the disclosure, the only co-solvent is lactic acid.

The disclosure further includes processes for preparing compositions of the disclosure as well as compositions of the disclosure made by those processes.

In some embodiments of the disclosure, compositions comprising one or more of sorbitol, such as D-(-)-Sorbitol, propylene glycol, polyethylene glycol, lactic acid, and phosphoric acid, such as orthophosphoric acid, and water are provided. In these and other embodiments, compositions comprising (a) caffeine citrate and sorbitol, such as D-(-)-Sorbitol; or (b) caffeine citrate and water; or (c) caffeine citrate and glycerol such as a 50/50 weight mix of glycerol in water; or (d) caffeine citrate and polyethylene glycol; or (e) caffeine citrate and propylene glycol; or (f) caffeine citrate and lactic acid, including 85% or greater lactic acid by weight; or (g) caffeine citrate and phosphoric acid, such as orthophosphoric acid, including such orthophosphoric acid that is 85% by weight, are provided.

In some embodiments, a composition comprising approximately equivalent amounts by weight of propylene glycol, D-(-)- Sorbitol/water (50/50), polyethylene glycol, glycerol/water (50/50), 85% lactic acid and 85% orthophosphoric acid is combined with about 57% water to make a solvent/co-solvent solution. In such embodiments, up to about 165 mg of caffeine citrate may be present at room temperature. In other embodiments, up to about 150 mg of caffeine citrate may be present in a solution of about 10.2 ml of water and about 9.8 ml of lactic acid.

The solubility of caffeine citrate in the compositions of the disclosure varies. The solubility measurements range from about 5 mg/ml to about 248 mg/ml. In these and other embodiments, the solubility of the compositions of the disclosure are above 100 mg/ml, above 200 mg/ml or between about 200 mg/ml and about 300 mg/ml. The higher the solubility of caffeine citrate in the solution, the less volume of solution needed to dose to a patient. For example, for a 15 mg/Kg dose for a 100 kg patient, 1500 mg caffeine citrate are required. Caffeine itself has been measured to have a solubility of 16 mg/ml (Sigma Aldrich). This translates to 753 mg caffeine. The solubility of caffeine citrate in water is about 56 mg/ml as measured herein, so about 27 ml of solution are needed. At a solubility of about 165 mg/ml, this reduces o about 9.2 ml. At a solubility of about 250 mg/ml, this reduces further about 6.1 ml. In other embodiments the volume of caffeine citrate in solution is between about 3 ml and about 7 ml. In some embodiments, the volume of caffeine citrate is about 5 ml. In these and other embodiments said volumes of caffeine citrate solution may be prepared as pharmaceutical preparations and delivered to patients to accelerate emergence from anesthesia.

In many embodiments, the compositions of the disclosure comprise caffeine citrate and lactic acid wherein the percent by mass of a lactic acid is between about 44.7% and about 45.3%, the percent by mass of water is between about 54.0% and about 54.6%, and the mass percent of caffeine citrate is between about 0.4% and about 1.0%. In these and other embodiments, the percent by mass of lactic acid can be between about 44.8% and about 45.2%, the mass percent of water can be between about 54.1% and about 54.5%, and the mass percent of caffeine citrate can be between about 0.5% and about 0.9%. Such embodiments further include a composition comprising about 45.0% lactic acid by mass, about 54.3% water by mass and about 0.70% caffeine citrate by mass.

Uses of caffeine citrate further include as treatments for headaches including migraine headaches, apnea, and improved asthma function. Accordingly, the caffeine citrate of the disclosure may be used in therapeutically effective amounts to treat headaches including migraine headaches, apnea, and asthma in patients in need thereof.

Other applications include as caffeine for beverages and food such as confectionaries. Because caffeine is bitter, reducing the volume of caffeine in a food product allows for increased volume of sweeteners or other non-bitter components so as to improve overall flavor.

The disclosure may be further described by one or more of the non-limiting clauses that follow.

Clause 1. A composition comprising caffeine citrate and one or more co-solvents.

Clause 2. The composition of clause 1 further comprising water.

Clause 3. The composition of any one of clauses 1-2, wherein at least one co-solvent is an organic acid.

Clause 4. The composition of any one of clauses 1-3, wherein at least one co-solvent is an alcohol.

Clause 5. The composition of clause 4, wherein the at least one co-solvent is a diol.

Clause 6. The composition of clause 4, wherein the at least one co-solvent is a triol.

Clause 7. The composition of clause 4, wherein the at least one co-solvent is a sugar alcohol.

Clause 8. The composition of any one of clauses 1-7, wherein at least one co-solvent is an inorganic acid.

Clause 9. The composition of any one of clauses 3-7, wherein the organic acid is an amino acid.

Clause 10. The composition of any one of clauses 3-7, wherein the organic acid is lactic acid.

Clause 11. The composition of clause 7, wherein the sugar alcohol is sorbitol.

Clause 12. The composition of clause 11, wherein the sorbitol is D-(-)-Sorbitol.

Clause 13. The composition of any one of clauses 4-5, wherein the alcohol is propylene glycol.

Clause 14. The composition of clause 4, wherein the alcohol is polyethylene glycol.

Clause 15. The composition of clause 6, wherein the triol is glycerin.

Clause 16. The composition of any one of clauses 1-2, wherein one or more co-solvents are chosen from an organic acid, an alcohol, or an inorganic acid.

Clause 17. The composition of clause 16, wherein the organic acid is lactic acid.

Clause 18. The composition of any one of clauses 16-17, wherein the alcohol is a sugar alcohol.

Clause 19. The composition of clause 18, wherein the sugar alcohol is sorbitol.

Clause 20. The composition of clause 19, wherein the sugar alcohol is D-(-)-Sorbitol.

Clause 21. The composition of any one of clauses 16-20, wherein the organic acid is an amino acid.

Clause 22. The composition of any one of clauses 16, 17, or 21, wherein the alcohol is a diol or triol.

Clause 23. The composition of clause 22, wherein the alcohol is a diol.

Clause 24. The composition of clause 23, wherein the alcohol is propylene glycol.

Clause 25. The composition of clause 22, wherein the alcohol is a triol.

Clause 26. The composition of clause 25, wherein the alcohol is glycerin.

Clause 27. The composition of any one of clauses 16-17, or 21 wherein the alcohol is polyethylene glycol.

Clause 28. The composition of any one of clauses 16-27, wherein the inorganic acid is phosphoric acid.

Clause 29. The composition of clause 28, wherein the phosphoric acid is orthophosphoric acid.

Clause 30. The composition of any one of clauses 16-29, further comprising water.

Clause 31. The composition of any one of clauses 1-30, further comprising a buffer.

Clause 32. The composition of any one of clauses 1-30, wherein the organic acid is not citric acid.

Clause 33. The composition of clause 31, wherein the buffer is a citrate buffer.

Clause 34. The composition of clause 33, wherein the buffer is citric acid and sodium citrate.

Clause 35. The composition of any one of clauses 1-34, further comprising a pharmaceutically acceptable excipient.

Clause 36. The composition of any one of clauses 1, 16, 30-35 wherein the co-solvent is lactic acid.

Clause 37. The composition of clause 36, wherein the only co-solvent is lactic acid.

Clause 38. The composition of 37, further comprising water.

Clause 39. The composition of any one of clauses 3-38, wherein the organic acid is a C1-C5 organic acid.

Clause 40. A method of making a composition comprising caffeine citrate, water and a co-solvent comprising the steps of combining one or more co-solvents with caffeine citrate, or a composition made by the method.

Clause 41. A composition comprising caffeine citrate and one or more of sorbitol, propylene glycol, polyethylene glycol, glycerin, lactic acid, and orthophosphoric acid.

Clause 42. The composition of clause 41, further comprising water.

Clause 43. A solution composition comprising caffeine citrate water, and one or more co-solvents wherein the solubility of caffeine citrate in the solution composition is greater than about 100 mg/ml solution.

Clause 44. The solution of clause 43, wherein the solubility of caffeine citrate in the solution composition is greater than about 200 mg/ml solution.

Clause 45. The solution of clause 44, wherein the solubility of caffeine citrate in the solution composition is between about 200 mg and about 300 mg/ml of solution.

Clause 46. A method of delivering a composition of any one of clauses 1-45 to a human patient comprising the steps of preparing a solution of any one of clauses 1-45 and administering said solution to said human.

Clause 47. The method of clause 46, wherein the administration is parenteral.

Clause 48. The method of clause 46, wherein the administration is intravenous.

Clause 49. The method of any one of clauses 46-48, wherein the administration is through a bolus dose.

Clause 50. The method of any one of clauses 46-48, wherein the administration is through a drip line.

Clause 51. The method of any one of clauses 46-50, wherein the administration occurs concurrently with the administration of an anesthesia agent to the patient.

Clause 52. The method of any one of clauses 46-50, wherein the administration occurs after the delivery of an anesthesia agent to the patient.

Clause 53. The method of any one of clauses 46-50, wherein the administration occurs before the delivery of an anesthesia agent to the patient.

Clause 54. A method of accelerating emergence from anesthesia comprising administering to a patient undergoing a medical procedure an anesthesia agent a pharmaceutically effective amount of a composition of any one of clauses 1-45.

Clause 55. The method of clause 54, wherein the administration is through a bolus dose.

Clause 56. The method of clause 54, wherein the administration is through a drip line.

Clause 57. The method of clause 54, wherein the administration occurs concurrently with the administration of the anesthesia agent to the patient.

Clause 58. The method of clause 54, wherein the administration occurs after the delivery of the anesthesia agent to the patient.

Clause 59. The method of clauses 54, wherein the administration occurs before the delivery of the anesthesia agent to the patient.

Clause 60. The method of any one of clauses 51-59, wherein the anesthesia agent is propofol.

Clause 61. The method of any one of clauses 51-59 wherein the anesthesia agent is one or more of isoflurane, sevoflurane, desflurane, propofol, or fentanyl.

Clause 62. The method of any one of clauses 54-61, wherein the emergence from anesthesia is at least 15 minutes faster than without the use of a composition of any one of clauses 1-45.

Clause 63. The method of any one of clauses 54-61, wherein the emergence from anesthesia is at least 30 minutes faster than without the use of a composition of any one of clauses 1-45.

Clause 64. The composition of any one of clauses 1-45, wherein the composition is a solution.

Clause 65. The composition of clause 64, wherein the volume of solution is between about 3 and about 7 ml.

Clause 66. The composition of clause 65, wherein the volume of solution is about 5 ml.

Clause 67. A composition comprising a solution of lactic acid, caffeine citrate, and water.

Clause 68. The composition of clause 67, wherein the lactic acid is a solution.

Clause 69. The composition of any one of clauses 67-68 wherein the lactic acid is a solution of greater than 85% lactic acid by weight.

Clause 70. The composition of any one of clauses 67-69, comprising between about 44.7% and about 45.3% lactic acid, between about 54.0% and about 54.6% water, and between about 0.4% and about 1.0% caffeine citrate.

Clause 71. The composition of clause 70, comprising between about 44.8% and about 45.2% lactic acid, between about 54.1% and about 54.5% water, and between about 0.5% and about 0.9% caffeine citrate.

Clause 72. The composition of clause 70, comprising about 45.0% lactic acid, about 54.3% water, and about 0.70% caffeine citrate.

Clause 73. The composition of clause 69, wherein the lactic acid solution is about 88.47% by weight lactic acid.

Clause 74. The composition of any one of clauses 69 or 73 wherein the lactic acid is in aqueous solution.

Clause 75. A method of treating headache, apnea or asthma comprising administering to a patient in need thereof a therapeutically effective amount of a composition of any one of clauses 1-39, 41-45, or 64-74.

Clause 76. The method of clause 75, wherein the headache is a migraine headache.

Clause 77. Use of a caffeine citrate composition of any one of clauses 1-39, 41-45, or 64-74 for accelerating the emergence from anesthesia.

Clause 78. Use of a caffeine citrate composition of any one of clauses 1-39, 41-45, or 64-74 in the preparation of a medicament comprising a therapeutically effective amount of caffeine citrate.

Clause 79. Use of a caffeine citrate composition of any one of clauses 1-39, 41-45, or 64-74 for the treatment of headaches, asthma, or apnea.

Clause 80. A beverage comprising a caffeine citrate composition of any one of clauses 1-39, 41-45, or 64-74.

Clause 81. A food comprising a caffeine citrate composition of any one of clauses 1-39, 41-45, or 64-74.

### EXAMPLES

### Example 1 - Material, Equipment, and Software

The following material, equipment, and software were used in the examples of the disclosure.

Materials:
Caffeine, citrated (Acros, lot A0404901), (also known as Caffeine citrate)
Distilled water (Meijer, lot 39-222, exp Oct. 31, 2020), (also known as Water)
D-(-)-Sorbitol ≥96%, High Purity Grade (VWR lot 19D0356470), (also known as Sorbitol)
(±)-1,2-Propanediol ≥99.5% (dry basis), BAKER ANALYZED ACS (JT Baker, batch no: 0000187716), (also known as Propylene glycol)
Polyethylene glycol 200 (Alfa Aesar, lot 10214220), (also known as Polyethylene glycol)
Glycerine ≥99.7%, Laboratory Reagent (VWR lot 19D0356528), (also known as Glycerol)
Lactic acid in aqueous solution ≥85% ACS Reagent (EMD Millipore Corp, lot 56169630), (also known as Lactic acid)
Orthophosphoric acid ≥85%, HiPerSolv CHROMANORM for HPLC (VWR lot 19A184013), (also known as orthophosphoric acid)

Equipment:
Denver Instrument Company A-250 balance
Eppendorf Research 1000 pipette, range 0.100 - 1.000 ml
Sonicator (Model O Conx. Jewelry Cleaner)
Escali model PRS00S scale
Mettler Toledo AG104 balance

Software:
Minitab 19
Excel version 1911

### Example 2- Experimental Design

Minitab 19 was used to create a design of experiments (DOE); specifically, a mixture design. An augmented Simplex Lattice Design for 7 factors was selected as a screening DOE. Such a design resulted in 15 experiments (runs) to be conducted. Each run tested a different co-solvent or mixture of co-solvents. The co-solvents tested in this experiment were each assigned a letter, A-G. The numbers in Table 1 represent the % of each co-solvent in each run.

Table 1

Screening DOE (numbers represent %)

| | Water | D-sorbitol in water (50/50 by weight) | Propylene glycol | Polyethylene glycol | Glycerol in water (50/50 by weight) | Lactic acid 85% | Ortho-phosphoric acid 85% |
|---|---|---|---|---|---|---|---|
| Run | A | B | C | D | E | F | G |
| 1 | 7.14 | 57.14 | 7.14 | 7.14 | 7.14 | 7.14 | 7.14 |
| 2 | 14.29 | 14.29 | 14.29 | 14.29 | 14.29 | 14.29 | 14.29 |
| 3 | 7.14 | 7.14 | 7.14 | 7.14 | 7.14 | 57.14 | 7.14 |
| 4 | 0 | 100 | 0 | 0 | 0 | 0 | 0 |
| 5 | 100 | 0 | 0 | 0 | 0 | 0 | 0 |
| 6 | 7.14 | 7.14 | 7.14 | 7.14 | 7.14 | 7.14 | 57.14 |
| 7 | 0 | 0 | 0 | 0 | 100 | 0 | 0 |
| 8 | 7.14 | 7.14 | 7.14 | 57.14 | 7.14 | 7.14 | 7.14 |
| 9 | 7.14 | 7.14 | 7.14 | 7.14 | 57.14 | 7.14 | 7.14 |
| 10 | 0 | 0 | 0 | 100 | 0 | 0 | 0 |
| 11 | 0 | 0 | 100 | 0 | 0 | 0 | 0 |
| 12 | 0 | 0 | 0 | 0 | 0 | 100 | 0 |
| 13 | 0 | 0 | 0 | 0 | 0 | 0 | 100 |
| 14 | 7.14 | 7.14 | 57.14 | 7.14 | 7.14 | 7.14 | 7.14 |
| 15 | 57.14 | 7.14 | 7.14 | 7.14 | 7.14 | 7.14 | 7.14 |

### Example 3 - Experimental Setup and Procedure

For each run involving a mixture of two or more co-solvents, 20 ml of co-solvent or co-solvent mixture was created by using the Eppendorf pipette to add the appropriate percentage of each individual co-solvent to a 20 ml scintillation vial. The exact ml of each co-solvent added to create the mixtures are listed in Table 2.

Table 2

Screening DOE (numbers represent ml)

| | Water | D-sorbitol in water (50/50 by weight) | Propylene glycol | Polyethylene glycol | Glycerol in water (50/50 by weight) | Lactic acid 85% | Ortho-phosphoric acid 85% |
|---|---|---|---|---|---|---|---|
| Run | A | B | C | D | E | F | G |
| 1 | 1.428 | 11.428 | 1.428 | 1.428 | 1.428 | 1.428 | 1.428 |
| 2 | 2.858 | 2.858 | 2.858 | 2.858 | 2.858 | 2.858 | 2.858 |
| 3 | 1.428 | 1.428 | 1.428 | 1.428 | 1.428 | 11.428 | 1.428 |
| 6 | 1.428 | 1.428 | 1.428 | 1.428 | 1.428 | 1.428 | 11.428 |
| 8 | 1.428 | 1.428 | 1.428 | 11.428 | 1.428 | 1.428 | 1.428 |
| 9 | 1.428 | 1.428 | 1.428 | 1.428 | 11.428 | 1.428 | 1.428 |
| 14 | 1.428 | 1.428 | 11.428 | 1.428 | 1.428 | 1.428 | 1.428 |
| 15 | 11.428 | 1.428 | 1.428 | 1.428 | 1.428 | 1.528 | 1.428 |

For sorbitol and glycerol, the 50/50 mixtures noted above were used to create any mixtures requiring those materials.

Approximately 50 mg of caffeine citrate was weighed into several 20 ml scintillation vials for use in the solubility experiments. Vials of caffeine citrate were created, as described in Table 3.

Table 3

Amount of caffeine citrate in each numbered vial

| Vial number | Caffeine citrate (mg) |
|---|---|
| 1 | 50.8 |
| 2 | 50.0 |
| 3 | 49.5 |
| 4 | 50.6 |
| 5 | 50.0 |
| 6 | 50.0 |
| 7 | 50.2 |
| 8 | 49.7 |
| 9 | 50.4 |
| 10 | 50.5 |
| 11 | 49.8 |
| 12 | 50.0 |
| 13 | 50.1 |
| 14 | 49.9 |
| 15 | 50.7 |
| 16 | 50.4 |
| 17 | 49.4 |
| 20 | 50.4 |
| 21 | 50.5 |
| 22 | 50.5 |
| 23 | 49.9 |
| 24 | 50.7 |
| 26 | 49.4 |
| 27 | 50.5 |
| 28 | 49.0 |
| 29 | 50.7 |
| 30 | 49.3 |
| 31 | 148.7 |

Several solubility experiments were conducted using various different parameters (how much co-solvent or water to add, sonication time, etc.). Ultimately, it was decided to add co-solvents or water in aliquots ranging from 0.100 to 1.000 depending on the observed solubility response. After adding each aliquot, the sample was sonicated for 30 minutes. If all the caffeine citrate had not dissolved (visual inspection), an additional aliquot was added, and the sample was sonicated for an additional 30 minutes. This procedure was repeated until the entire sample dissolved. Solubility was determined based on visual inspection.

For D-sorbitol (a solid), it was dissolved in water prior to use for run #4. Glycerol was used as-is for run #7, but it was very viscous and difficult to pipette. An approximate 50/50 mixture of glycerol and water was made and run #7 was repeated with this mixture. For use in creating various solvent mixtures, 50/50 by weight solutions of sorbitol in water, and glycerol in water, were made and used.

### Example 4- Initial Solubility Results

The results are presented in Table 4.

Table 4

Initial solubility results

| Run number | Vial number | Caffeine citrate (mg) | Solvent | Solvent added until dissolution (ml) | Solubility (mg/ml) |
|---|---|---|---|---|---|
| 1 | 1 | 50.8 | Solution 1 | 2.000 | 25.4 |
| 2 | 2 | 50.0 | Solution 2 | 2.000 | 25.0 |
| 3 | 3 | 49.5 | Solution 3 | 1.500 | 33.0 |
| 4 | 22 | 50.5 | Sorbitol in water | 6.500 | 7.8 |
| 5 | 21 | 50.5 | Water | 0.900 | 56.1 |
| 6 | 6 | 50.0 | Solution 6 | 2.000 | 25.0 |
| 7 | 28 | 49.0 | Glycerol in water | 4.000 | 12.3 |
| 8 | 8 | 49.7 | Solution 8 | 10.000 | 5.0 |
| 9 | 9 | 50.4 | Solution 9 | 2.000 | 25.2 |
| 10 | 24 | 50.7 | Polyethylene glycol | 10.000 | 5.1 |
| 11 | 23 | 49.9 | Propylene glycol | 10.000 | 5.0 |
| 12 | 26 | 49.4 | Lactic acid 85% | 5.000 | 9.9 |
| 13 | 27 | 50.5 | Orthophosphoric acid 85% | 2.000 | 25.3 |
| 14 | 14 | 49.9 | Solution 14 | 4.500 | 11.1 |
| 15 | 17 | 49.4 | Solution 15 | 0.300 | 164.7 |

One of the tests (solution 15) surpassed the target of 150 mg caffeine citrate per ml of solution.

### Example 5 - Creating a DOE Model

The solubility results from Table 4 were entered into the DOE model in Minitab. The model was adjusted, using statistical evaluation tools as guidance, until only factors with P-values statistically significant (<0.05) remained in the model. The following terms remained in the model and are listed below, along with their coefficient values. A large positive number represents a synergistic impact on solubility whereas a large negative number represents an antagonistic relationship.
Interaction term of water and sorbitol: 1672
Interaction term of water and propylene glycol: 1556
Water: 56.6
Sorbitol: 25.8
Orthophosphoric acid: 22.2
Lactic acid: 13.2
Glycerol: 11.9
Propylene glycol: 5.5
Polyethylene glycol: -1.9
Interaction term of sorbitol and propylene glycol: -1914

### Example 6 - Results of DOE Modeling

Using the adjusted model, the “maximize response” function was used to predict a solvent mixture that would have the highest solubility, based on data entered into the model. That mixture suggested adding 10.235 ml of water with 9.765 ml of lactic acid. This mixture was created and used to dissolve 148.7 mg of caffeine citrate, adding 0.1 to 0.2 ml aliquots at a time and sonicating for 30 minutes after the addition of each aliquot. Ultimately, all the caffeine citrate dissolved in 0.6 ml of this new solvent mixture, resulting in a calculated solubility of 247.8 mg/ml.

### Example 7 - Three Component Solubility Experiments and Limit Tolerance Measurement

Solutions of caffeine citrate, lactic acid, and sodium hydroxide were created and the solubility was measured. Several of the solutions had a solubility greater than 150 mg/mL with the highest being 254 mg/mL. Prior to combining with caffeine citrate, a combination solution was prepared by combining solutions of 1M NaOH and a solution of >85% (88.47%) lactic acid. 1M NaOH was made by adding solid NaOH to water. The mass percents below in Table 5 represent the mass percent of water, NaOH, and lactic acid in the combination solution prior to contact with caffeine citrate. Once combined a volume of the solution shown in Table 5 below was added to caffeine citrate until the caffeine citrate dissolved (“Solution added (mL)” column). The solubility was determined by dividing the mass of caffeine citrate by the volume of combined solution added. The pH was measured of the combined solution as well as the solution with the dissolved caffeine citrate.

Table 5

Further Solubility Results

| %Lactic acid | NaOH | %Water | pH | Caffeine citrate (mg) | Solution added (mL) | Solubility (mg/mL) | pH of dissolved sample |
|---|---|---|---|---|---|---|---|
| 80.19% | 0.36% | 19.45% | 0.93 | 50.7 | 0.750 | 67.6 | 0.93 |
| 45.86% | 1.85% | 52.29% | 2.43 | 50.7 | 0.200 | 254 | 2.2 |
| 63.27% | 1.09% | 35.63% | 1.77 | 50.3 | 0.300 | 168 | 1.69 |
| 27.92% | 2.63% | 69.45% | 2.98 | 50.5 | 0.600 | 84.2 | 2.92 |
| 9.45% | 3.43% | 87.12% | 9.26 | 49.4 | 3.050 | 16.2 | 4.65 |
| 48.82% | 1.72% | 49.46% | 2.6 | 50.5 | 0.20 | 253 | 2.23 |

When taking into account typical tolerance limits of lactic acid and NaOH as set by regulatory authorities or suggested in the literature (not more than 32.3% lactic acid and not more than 13% NaOH), an additional experiment was performed using 51.8 mg caffeine citrate containing 32.2% lactic acid by mass and no NaOH which resulted in a solution that had a solubility of 259 mg/mL. In that experiment, a 0.2 mL aliquot of a 23.453 mL of water and 12.170 mL of 88.47% lactic acid (density of 1.1 g/mL) was combined with a vial containing caffeine citrate whereby the caffeine citrate had fully dissolved after 30 minutes.

1. A composition comprising caffeine citrate and one or more co-solvents.

2. The composition of claim 1 further comprising water.

3. The composition of claim 1, wherein at least one co-solvent is an organic acid.

4. The composition of claim 1, wherein at least one co-solvent is an alcohol.

5-7. (canceled)

8. The composition of claim 1, wherein at least one co-solvent is an inorganic acid.

9. (canceled)

10. The composition of claim 3, wherein the organic acid is lactic acid.

11-30. (canceled)

31. The composition of claim 1, further comprising a buffer.

32. (canceled)

33. The composition of claim 31, wherein the buffer is a citrate buffer.

34-42. (canceled)

43. A solution composition comprising caffeine citrate, water , and one or more co-solvents wherein the solubility of caffeine citrate in the solution composition is greater than about 100 mg/ml solution.

44. The solution of claim 43, wherein the solubility of caffeine citrate in the solution composition is greater than about 200 mg/ml solution.

45. The solution of claim 44, wherein the solubility of caffeine citrate in the solution composition is between about 200 mg/ml and about 300 mg/ml of solution.

46-53. (canceled)

54. A method of accelerating emergence from anesthesia comprising administering to a patient undergoing a medical procedure with an anesthesia agent a pharmaceutically effective amount of a composition of claim 1.

55-59. (canceled)

60. The method of claim 54, wherein the anesthesia agent is propofol.

61. The method of claim 54, wherein the anesthesia agent is one or more of isoflurane, sevoflurane, desflurane, propofol, or fentanyl.

62. The method of claim 54, wherein the emergence from anesthesia is at least 15 minutes faster than without the use of the composition .

63. (canceled)

64. The composition of claim 1, wherein the composition is a solution.

65-66. (canceled)

67. A composition comprising a solution of lactic acid, caffeine citrate, and water.

68-69. (canceled)

70. The composition of claim 67, comprising between about 44.7% and about 45.3% lactic acid, between about 54.0% and about 54.6% water, and between about 0.4% and about 1.0% caffeine citrate.

71. The composition of claim 70, comprising between about 44.8% and about 45.2% lactic acid, between about 54.1% and about 54.5% water, and between about 0.5% and about 0.9% caffeine citrate.

72-74. (canceled)

75. A method of treating headache, apnea or asthma comprising administering to a patient in need thereof a therapeutically effective amount of a composition of claim 1.

76-81. (canceled)
